(19) United States
(12) Patent Application Publication
(10) Pub. No.: US 2009/0259148 A1
(43) Pub. Date: Oct. 15, 2009

(54) HEALTH MANAGEMENT DEVICE

(75) Inventors: Richard Daniel Willmann, Siegburg (DE); Gerd Lanfermann, Aachen (DE); Edwin Gerardus Johannus Maria Bongers, Roermond (NL); Juergen Te Vrugt, Aachen (DE)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/373,756

(22) PCT Filed: Jul. 5, 2007
(86) PCT No.: PCT/IB2007/052639

§ 371 Date: Jan. 14, 2009

(30) Foreign Application Priority Data
Jul. 19, 2006 (EP) 06117476.9

(51) Int. Cl. A61B 5/11

(52) U.S. Cl. 600/595

(57) ABSTRACT

The invention relates to a health management system comprising a body or limb movement detecting means for detecting the movements and position of a users body or limb(s) in 3D space, a movement analyzing means for analyzing the data of the measurement carried out by the body or limb movement detecting means, wherein the body or limb movement detecting means comprises at least three sensors or markers for tracking a user's body or limb movement in 3D space by measuring an angle embedded by two body parts of the user which are connected to each other by a joint being the apex of the angle to be measured, at which one of the sensors or markers is provided. To detect the offset a change in distance between two neighboring sensors or markers indicates an offset of the sensor at the joint spaced apart from the apex.

The present invention relates to a system and a method for rehabilitation and/or physical therapy for the treatment of neuromotor disorders, such as a stroke. After a stroke patients often suffer of disturbances in movement coordination. These disturbances are the least well understood but often the most debilitating with respect to functional recovery following brain injury. These deficits in coordination are expressed in the form of abnormal muscle synergies and result in limited and stereotype movement patterns that are functionally disabling. The result of these constraints in muscle synergies is for example an abnormal coupling between shoulder abduction and elbow flexion in the arm, which significantly reduces a stroke survivor reaching space when he/she lifts up the weight of the impaired arm against gravity. Current neurotherapeutic approaches to mitigate these abnormal synergies have produced limited functional recovery. In the leg the expression of abnormal synergies results in coupling hip/knee extension with hip adduction. The result of this is a reduced ability of activating hip abductor muscles in the impaired leg during stance.

When traditional therapy is provided in a hospital or rehabilitation center, the patient is usually seen for half-hour sessions, once or twice a day. This is decreased to once or twice a week in outpatient therapy.

Current studies indicate that motor exercising for improving the coordination of the patient can be done at home as part of a tele-rehabilitation solution. Available systems use videoconferencing approach, where the patient exercises in front of a camera at a time that is convenient for him. Such a system is for example disclosed in the US 2002/0146672 A1. This system includes a device, which senses the position of digits of a user's hand of the user while the user is performing an exercise by interacting with a virtual image. A second device provides feedback to the user and measures the position of the digits of the hand while the user is performing an exercise by interacting with a virtual image. The virtual image is updated based on targets determined for the user's performance in order to provide harder or easier exercises. Accordingly no matter how limited a users movement is, if the users performances falls within a determent parameter range, the user can pass the exercise trial and the difficulty level can gradually be increased.

The data of the user's performance is stored and reviewed by a therapist. Therefore, the rehabilitation system is distributed between a rehabilitation site, a data storage site and a data access site through an internet connection between the sites. The data access site includes software that allows a doctor/therapist to monitor the exercises performed by the patient in real time using a graphic image of the patient's hand, by sending the recorded videos to the doctor or physiotherapist, who reviews the exercises and gives feedback. There are a number of passive and active devices, e. g. Theraband or Reck MotoMed, that allow a user to perform such exercising at home as part of a tele-rehabilitation solution.

One of the most prominent disabilities stroke survivors suffer from is half sided paralysis of the upper limbs. Rehabilitation exercises are proven to be efficient in regaining motor control, provided the training is intense and the patient is guided in the therapy. Technical solutions for unsupervised home stroke rehabilitation require the use of markers or sensors for acquiring the patient's posture during exercises.

A very attractive sensor solution is using cameras, which view 2D or 3D coordinates of limbs and joints in space, depending on whether a single or multi camera system is used. However, acquiring limb position from a camera position requires finding and tracking of limbs in the image, which is a non-trivial task and an unsolved problem today, if no markers are used (see e.g. “the evolution of methods for the capture of human movement leading markerless motion capture for bio medical applications”, i.g. Mundermann et al., J. Neuro Engineering and Rehabilitation 2006, 3:6).

The tracking of marker positions by cameras in both the optical range and in the infrared is very reliable. In this area, a lot of commercial products exist.

The problem with such an approach is that existing marker-based tracking systems assume the user to be skilled enough to place the markers at exactly reproducible places; thus consistent results should be achieved. This assumption becomes unrealistic, if the user is a stroke victim. Instead, the exact position of the markers on the limbs will differ from one use to the other, since the user is not able to fix the marker or sensor in exactly the same position because of a loss of control of the movement of his arms hands and/or fingers.

It is therefore an object of the present invention to provide a system and a method that ensures proper functionality of the system even in the event of inaccurate placing of the markers or sensors on the user's limb.

This object is solved by a system and a method according to claims 1 and 7.

The health management system according to the invention comprises a body or limb movement detecting means for detecting the movements of a users body or limb(s), a movement analyzing means for analyzing the data of the measurement carried out by the body or limb movement detecting means, wherein the body or limb movement detecting means comprises at least three markers for tracking a user's body or limb movement. To analyse the movement an angle between two body parts of the user, which are connected to each other by a joint, is measured. The joint builds the apex of the angle to be measured, at which one of the markers is provided.

To determine whether the marker at the joint has been placed in exactly the right position the distance of two neighboring markers on the user's limbs is measured. A change in distance between two neighboring sensors or markers indicates an offset of the sensor at the joint spaced apart from the apex of the angle.

For calculation or estimation of the offset of the marker at the joint the movement analyzing means may include an automatic motor learning program, wherein the motor learning program includes an algorithm following the equation:

x=argmin_{{0<x}}(SUM_{{t=1 . . . T}}(Lₜ²)−SUM_{{t=1 . . . T}}²(Lₜ)),

where Lₜ=(1+x) (R_{Marker3}−R_{Marker2}).

With this algorithm the joint angle from the position of the markers (R_{Marker3},R_{Marker2}) on the limbs is assessed by estimating a first offset (x) and adjusting the assumption by analyzing the user's motion (see also FIG. 2). The current output of the markers or sensors indicates a decrease of change in distance between the two neighboring sensors until the marker offset converged to a value that is within a measurement accuracy of the true value of the marker offset (x=o).

Thus the system gives the user the freedom to place the markers on his limbs with a great degree of freedom and still to receive sensible system behavior.

The automatic motor learning program may select the initial offset range as a subsequent target offset range for each following series of measurements in which said predetermined success criteria is not met and the current output of the sensor units may indicate a decrease of the change in distance between two neighboring sensors.

An alternative embodiment of the present invention provides instead of the automatic motor learning program a program which upon a measurement of an offset of the marker at the joint generates a stimulation signal for causing the user to move the sensor towards the apex of the angle build between the limbs of the user to minimize the offset of the marker at the joint.

The body or limb movement measuring means may be at least one camera-based computer vision with markers or markers motion tracking by computer vision and/or one inertial sensors, at least one sensor garment and/or any other motion or position sensor. Markers can either be colour markers or retro-reflective IR-markers depending on which cameras are used.

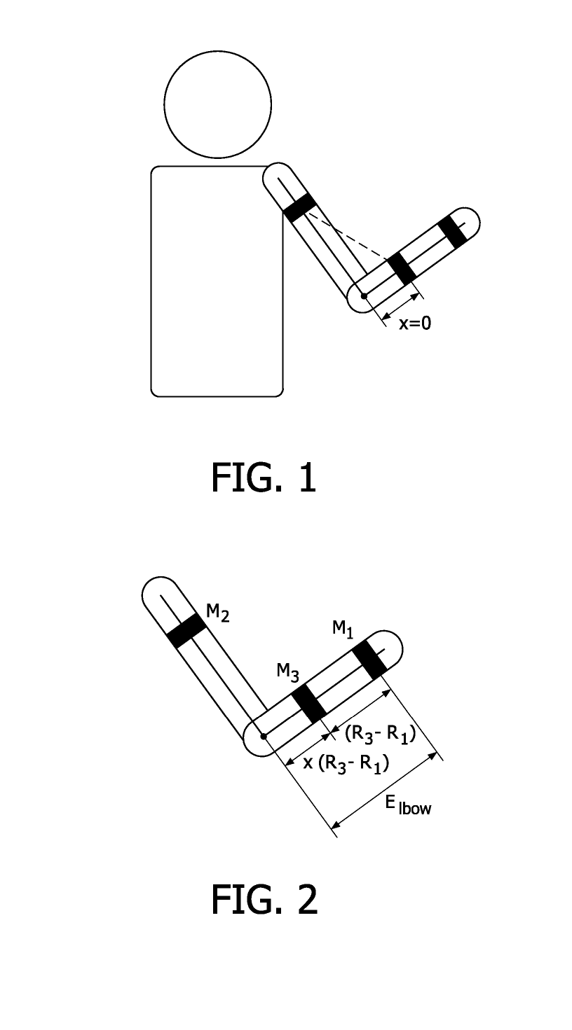

A system, which meets the above mentioned objects and provides other beneficial features in accordance with the presently preferred exemplary embodiment of the invention will be described below with reference to FIGS. 1 to 3. Those skilled in the art will readily appreciate that the description given herein with respect to those figures is for explanatory purposes only and is not intended in any way to limit the scope of the invention.

FIG. 1 shows the change of an angle enclosed of an upper and a lower arm of the user;

FIG. 2 shows schematically the correlation of the angle and the placement of the markers or sensors;

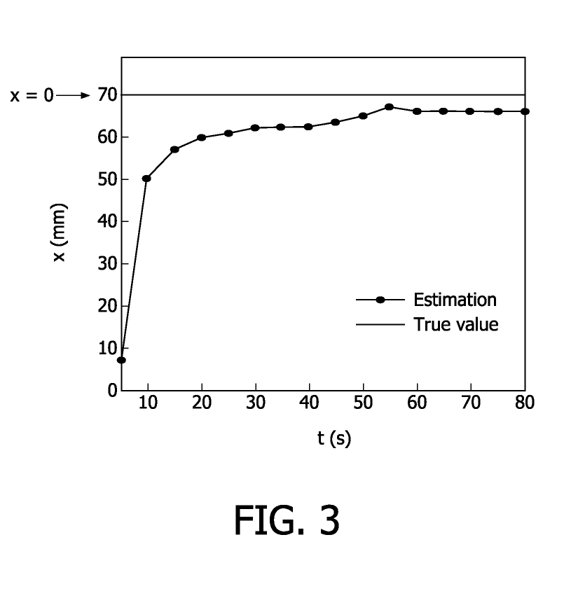

FIG. 3 shows an example of a marker offset learning curve.

As can be seen in FIG. 1 for the example of tracking two positions of the marker in the area of the joint are indicated with two different lines. In one case the marker or sensor is placed exactly at the joint so the angle build by the three sensors or markers is identical to the angle embedded by the upper and the lower arm. In the event of the second line the marker has been placed with an offset on the upper arm. Assuming that the elbow marker has been placed exactly at the joint with other words in the apex of the angle embedded by the upper and the lower arm leads to a wrong angle. If the sensor at the joint is positioned spaced apart form the apex on the upper arm, the angle build by the three sensors is bigger than the angle in case of an exact positioning of the sensor at the joint. On the other hand, if the sensor at the joint is spaced apart from the apex on the lower arm, the angle is smaller than the angle of an accurate positioning of a sensor.

To get the correct angle, the offset between the marker or the sensor and the joint has to be determined, which in the case sketched in FIG. 1 compared to the angle indicated leads to a smaller angle.

Therefore the system according to the invention analyzes the movement data and takes constraints of the human body into account. Thus the marker or sensor based tracking system becomes inured to a variation in putting on the markers or sensors.

For analyzing the movement data and taking constrains of the human body into account the health management system in one embodiment of the present invention includes a computer system with a CPU, storage and screen. To track the movement of the user a camera is provided in this embodiment. The camera may operate in the optical or infrared and is connected to the computer. Three markers are placed on a patient's limb, in this example at the user's arm. Markers or sensors can either be color markers or reflective markers depending on which type of camera is used. One sensor is placed on the user's wrist one on the upper arm and one in the area of the joint, in this case the elbow. Furthermore a storage for the acquired marker motion is provided.

After starting the computer program for estimating the patient's posture from marker-based camera images, an initial assumption is made that the offset between the joint and the marker is zero, which means that the marker is positioned at exactly the right position without any offset. Afterward the user starts moving and the system records the movement and adjusts the assumption on the marker offset iteratively by analyzing the motion.

Since there is no change in the angle or the relation of the sensors if the markers or sensors at the wrist or the upper arm are not placed exactly at the same position it doesn't matter if they are placed a bit higher or lower compared to an earlier use or measurement.

The only critical marker positioning is that of the marker at the joint of the limb to be detected. Therefore the distance between two neighboring markers or sensors is analyzed. If there is no change in distance between the neighboring markers the marker at the joint is placed at exactly the right position and the measurement can be started right away without any further adjusting steps.

A change of the distance between two neighboring markers however indicates the presence of an offset in the placing of the sensor at the joint. Now there are two possibilities in handling the offset.

One alternative instructs the user to move the marker at the joint in the direction of the joint. Therefore positioning means are provided at the fastening means of the marker, for example positioning screws that allow a user having difficulties in accurate moving his fingers a precise adjusting of the marker by driving the screw and thereby slowly and precisely moving the marker in the right direction towards the joint. If after an adjustment of the marker at the joint the change in distance gets bigger this is an indication that the marker has been moved in the wrong direction and the system may instruct the user to drive the screw in the other direction.

With the second embodiment a movement of the marker towards the joint is not even necessary. The offset of the marker is calculated and automatically integrated and recognized in the analysis of the movement of the user. In this case first of all the correlations between the motion of the marker on the upper arm and the marker in the area of the joint and of the marker on the lower arm or the wrist and the marker in the area of the joint have to be computed to find out if the marker at the joint is placed on the upper arm or on the lower arm.

As upper and lower arm are relatively rigid in itself a higher correlation is expected for markers on the same arm. So if for example a change in distance between two neighboring sensors or markers between the marker at the lower arm and the joint marker can be measured it indicates that the marker at the joint is placed on the other part of the arm in this example on the upper arm.

Once it is known at which arm the joint marker is placed the offset from the joint has to be estimated. Following the assumption above that the joint marker (marker 3) is placed on the upper arm the distance between the joint marker and the marker on the lower arm (marker 1) will vary depending on the movement of the arm, which leads to a change of the angle embedded by the upper and the lower arm, while the distance between the marker on the upper arm (marker 2) and the marker at the joint does not vary at all as the skeleton is rigid in this direction. Therefore the following algorithm to estimate the marker position on the limbs from body motion may be used:

The location of the joint—here the elbow—is given by (see also FIG. 2):

E_{lbow}=(1+x) (R_{Marker3}−R_{Marker2})

If the marker is on the lower arm the location is accordingly given by:

E_{lbow}=(1+x) (R_{Marker3}−R_{Marker2})

where x is the offset given as a fraction of the distance between markers 2 and 3 or in alternative 2 between markers 1 and 3. The approximation to correct x=o, wherein o is the real offset is found by minimizing the variation in the distance of expected joint position and wrist position as observed from the recorded motion by the following algorithm:

x=argmin_{{0<x}}(SUM_{{t=1 . . . T}}(Lₜ²)−SUM_{{t=1 . . . T}}²(Lₜ)),

where Lₜ=(1+x) (R_{Marker3}−R_{Marker2}).

The estimation of x improves over time as the SUM values then converge to the expectation values and becomes in the best way x=o.

The result of this marker offset over time can be seen in FIG. 3. After about a minute the marker offset converged to a value that is within the measurement accuracy of the estimation of the true value for the marker offset. With this cyclic and iterative approximation an automatic marker position learning has taken place. Thus the system gives the user the freedom to place the markers on his limbs with a great degree of freedom and still to receive sensible system behavior.

1. Health management system comprising:
a body or limb movement detecting means for detecting the movements and position of a users body or limb (s) in 3D space,
a movement analyzing means for analyzing the data of the measurement carried out by the body or limb movement detecting means;
wherein the body or limb movement detecting means comprises at least three sensors or markers for tracking a user's body or limb movement in 3D space by measuring an angle embedded by two body parts of the user which are connected to each other by a joint being the apex of the angle to be measured, at which one of the sensors or markers is provided, characterized in that a change in distance between two neighboring sensors or markers indicates an offset of the sensor at the joint spaced apart from the apex, the change in distance corresponding to the body or limb movement.

2. The health management system according to claim 1,
characterized in that from a measurement of an offset of the marker at the joint a stimulation signal is generated for causing the user to move the sensor towards the apex.

3. The health management system according to claim 1, wherein the body or limb movement measuring means is selected from a camera-based computer vision with markers, a sensor garments a motion sensor, and a position sensor.

4. The health management system according to claim 3, further comprising a least one mode stimulator that includes an audio mode stimulator having an audio stimulation unit or a video mode stimulator with a video stimulation unit.

5. (canceled)

6. (canceled)

7. A method of automatically position learning for camera-based limb tracking in particular in home stroke rehabilitation, comprising the steps of:
placing at least three markers on a user's limb to be analyzed for tracking a user's body or limb movement, so that they build an angle; embedded by two body parts of the user which are connected to each other by a joint being the apex of the angle to be measured, at which one of the markers is provided;
comparing positions of the markers relative to each other, wherein a change in distance between two neighboring markers indicates an offset of the marker at the joint of the limb spaced apart from the apex.

8. The method according to claim 7, characterized in that it further includes the steps of
computing the motion between the neighboring sensors to determine if the marker at the joint is placed at the upper or lower limb;
generating a first offset value assuming that offset between joint and marker is zero;
recording the user's movement and adjusting the assumption on the marker or sensor offset by analyzing the motion.

9. The method according to claim 8, characterized in that it further includes the step of
minimizing the variation in the distance of the markers to the expected joint position as observed from the recorded motion.

10. The method according to claim 7, characterized in that it comprises steps of
generating a visual and/or additive stimulation signal when said offset data is above a target offset range so as to cause the user to adjust the location of the sensor by the joint by moving the sensor towards the apex of the angle embedded between the user's limbs.
